(19) United States
(12) Patent Application Publication
(10) Pub. No.: US 2010/0097576 A1
(43) Pub. Date: Apr. 22, 2010

(54) Administering and analyzing ophthalmic examinatioins

(75) Inventors: Erwin Witt, Long Grove, IL (US); Keith A. Maslovitz, Arlington Heights, IL (US)

Correspondence Address: GREENBERG TRAURIG, LLP, 77 WEST WACKER DRIVE, SUITE 3100, CHICAGO, IL, 60601-1732, US

(73) Assignee: Woodlyn, Inc., Arlington Heights, IL (US)

(21) Appl. No.: 12/288,090

(22) Filed: Oct. 16, 2008

(51) Int. Cl. A61B 3/18

(52) U.S. Cl. 351/246; 351/200

(57) ABSTRACT

The present invention relates to technology for, among other things, administering, and analyzing the results of, ophthalmic examinations. The technology involves generating a display on a display device to enable a first ophthalmic exam to be administered, receiving a request for results of a second ophthalmic exam, requesting the results of the second ophthalmic exam from a data source, receiving the results of the second ophthalmic exam from the data source, displaying the results of the second ophthalmic exam on the display device and enabling annotation or modification of the results displayed on the display device.

### BACKGROUND

1. Field

Various embodiments of the present invention generally relate

2. Background

A typical trip to an ophthalmic office usually involves several different ophthalmic examinations for a patient—each of which often involves a separate testing apparatus. For example, the patient may receive a glaucoma exam using a non-contact tonometer, then a visual field exam using a perimeter, then a retinal exam using a Fundus camera, etc. Oftentimes, many of these testing apparatuses are found in physically separate locations within the ophthalmic practitioner's office. The apparatuses for performing pre-examinations, such as low-vision exams, color blindness exams, auto-refractions, and glaucoma exams, which may be performed by a technician, may be located in a “pre-test” room, while the apparatuses for performing other exams, such as refracting, retinoscopy, and gonioscopy, which may only be performed by a licensed optometrist or ophthalmologist, may be located in a separate examination room.

Thus, the typical patient-flow in an optometric clinic is as follows. First, the patient is taken to a pre-test room by a technician. There, the technician will usually perform those tests on the patient that do not require optometrist/ophthalmologist involvement. Once the first series of tests have been completed, the technician escorts the patient to an examination room, where the patient waits for the practitioner. While the patient is waiting, the technician provides the results of the first series of test to the ophthalmic practitioner, and the practitioner reviews and (sometimes) annotates the results.

Once the practitioner has reviewed the test results, the practitioner will then usually join the patient in the examination room, where the practitioner will complete the patient's eye examination. This will often involve the practitioner performing some additional tests on the patient, such as refracting, gonioscopy or retinoscopy.

Clearly, it is not desirable to leave the patient waiting for an extended period of time. Thus, the practitioner generally only has time to conduct a brief, cursory review of the results of the tests performed by the technician, and it is often desirable to review the results more thoroughly at a later time.

Conventionally, the results of the abovementioned tests were printed and inserted into the patient's file. If a practitioner wished to annotate the results, s/he would simply make written notes in the patient's file. Clearly, this is undesirable from the standpoint of efficiency and portability, since physical access to the patient's file is necessary in order to review and/or annotate test results.

More recently, testing apparatuses have allowed for electronic annotation of test results; however, such annotations are required to be made at the testing apparatus itself, before the results are printed or saved electronically to a static file format. This is not only inefficient, in that a practitioner must tie up a testing apparatus in order to annotate the results electronically, but the annotation interfaces on such testing apparatuses can also be cumbersome.

More recent advancements in optical testing equipment have focused on creating so-called “distributed” environments among the various testing apparatuses and throughout the associated clinics. In such environments, test results are saved to a centralized database that is accessible by other computers in communication therewith. One major drawback in conventional distributed environments is that these systems save the test results to the database as static image files, such as Joint Photographic Experts Group (JPEG) files, Tagged Image File Format (TIFF) files, Graphics Interchange Format (GIF) files, Bitmaps (BMP), Portable Document Format (PDF) files, or the like. It should be appreciated that such file formats are all essentially two-dimensional “snapshots” of the test results, and the viewer applications therefor only allow for a limited amount of file manipulation (i.e. zooming and two-dimensional panning). Thus, these formats are typically incapable of fully representing the three-dimensional data generated by some of today's more advanced eye testing technologies. For example, a corneal topographer creates a three-dimensional, topographical map of the surface of the eye which, in its native format, can be freely rotated so as to view the rendered image from a variety of perspectives. Clearly, such functionality is not achievable with a PDF, JPEG, or other such static image file. Thus, in conventional systems, the practitioner or technician must select the “best” snapshots of the three-dimensional data to save to the database.

Moreover, none of the technologies discussed above, no matter how recent, allows for the practitioner to present the patient's test results to the patient in a convenient, user-friendly, and full-featured manner. On the contrary, to do so using conventional technologies would require the practitioner and the patient to go back to the pre-test room containing the testing apparatus having the desired test results—during which time the testing apparatus is again unavailable for use by any other practitioner or technician.

### SUMMARY

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

Described herein is technology for, among other things, administering, and analyzing the results of, ophthalmic examinations. The technology involves generating a display on a display device to enable a first ophthalmic exam to be administered, receiving a request for results of a second ophthalmic exam, requesting the results of the second ophthalmic exam from a data source, receiving the results of the second ophthalmic exam from the data source, displaying the results of the second ophthalmic exam on the display device and enabling annotation or modification of the results displayed on the display device.

### BRIEF DESCRIPTION OF THE DRAWINGS

The accompanying drawings, which are incorporated in and form a part of this specification, illustrate embodiments of the invention and, together with the description, serve to explain the principles of embodiments of the invention:

FIG. 1 is a block diagram of an exemplary computing system environment for implementing various apparatuses used in conjunction with embodiments of the present invention;

FIG. 2 illustrates a block diagram of a system 200 for administering and analyzing ophthalmic examinations, in accordance with various embodiments of the present invention;

FIG. 3 illustrates an example exam room setup of an embodiment of the present invention;

FIG. 4 illustrates a smart ophthalmic examination apparatus displaying a visual acuity test, in accordance with various embodiments of the present invention;

FIG. 5 illustrates a smart ophthalmic examination apparatus displaying a first view of a three-dimensional graphical representation of the results of a corneal topography test, in accordance with various embodiments of the present invention;

FIG. 6 illustrates a smart ophthalmic examination apparatus displaying a rotated view of the three-dimensional graphical representation of FIG. 5, in accordance with various embodiments of the present invention;

FIG. 7 illustrates a smart ophthalmic examination apparatus displaying a annotated version of the three-dimensional graphical representation of FIG. 5, in accordance with various embodiments of the present invention;

FIG. 8 illustrates a smart ophthalmic examination apparatus displaying an exam room status indicator, in accordance with various embodiments of the present invention; and

FIG. 9 illustrates a smart ophthalmic examination apparatus displaying an updated exam room status indicator, in accordance with various embodiments of the present invention.

### DETAILED DESCRIPTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be apparent to one of ordinary skill in the art that the present invention may be practiced without these specific details. In other instances, well known methods, procedures, components, and circuits have not been described in detail as not to unnecessarily obscure aspects of the present invention.

Some portions of the detailed descriptions that follow are presented in terms of procedures, logic blocks, processing, and other symbolic representations of operations on data bits within a computer or digital system memory. These descriptions and representations are the means used by those skilled in the data processing arts to most effectively convey the substance of their work to others skilled in the art. A procedure, logic block, process, etc., is herein, and generally, conceived to be a self-consistent sequence of steps or instructions leading to a desired result. The steps are those requiring physical manipulations of physical quantities. Usually, though not necessarily, these physical manipulations take the form of electrical or magnetic signals capable of being stored, transferred, combined, compared, and otherwise manipulated in a computer system or similar electronic computing device. For reasons of convenience, and with reference to common usage, these signals are referred to as bits, values, elements, symbols, characters, terms, numbers, or the like with reference to the present invention.

It should be borne in mind, however, that all of these terms are to be interpreted as referencing physical manipulations and quantities and are merely convenient labels and are to be interpreted further in view of terms commonly used in the art. Unless specifically stated otherwise as apparent from the discussion herein, it is understood that throughout discussions of the present embodiment, discussions utilizing terms such as “determining” or “outputting” or “transmitting” or “recording” or “locating” or “storing” or “displaying” or “receiving” or “recognizing” or “utilizing” or “generating” or “providing” or “accessing” or “checking” or “notifying” or “delivering” or the like, refer to the action and processes of a computer system, or similar electronic computing device, that manipulates and transforms data. The data is represented as physical (electronic) quantities within the computer system's registers and memories and is transformed into other data similarly represented as physical quantities within the computer system memories or registers or other such information storage, transmission, or display devices.

### Overview

Generally speaking, embodiments provide for methods and systems that allow for an ophthalmic practitioner to utilize a first examination apparatus to review and annotate exam results not only from the first examination apparatus, but also from a second, separate examination apparatus. In a preferred embodiment, the first examination apparatus is a wall-mountable, flat-panel display, which is capable of displaying one or more ophthalmic exams, such as a visual acuity exam. Responsive to an input from an ophthalmic practitioner, the display can switch from an examination mode to an analysis mode, in which the user is able to review, manipulate and annotate the exam results from the second or other examination apparatuses.

### Exemplary Operating Environment

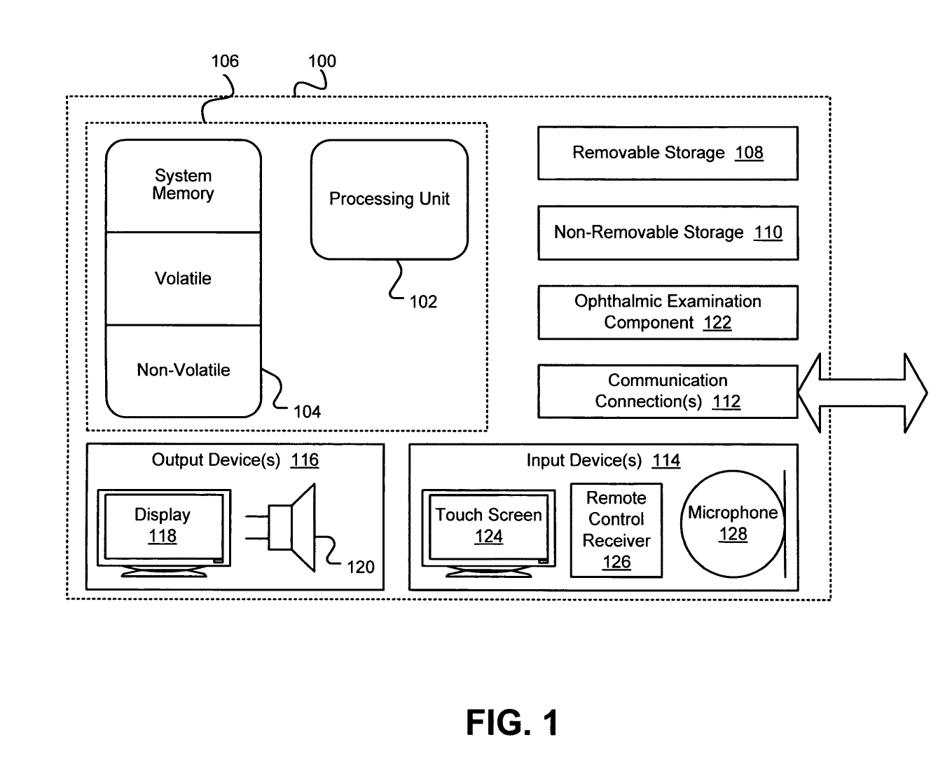

With reference to FIG. 1, an exemplary system for implementing various apparatuses used in conjunction with embodiments of the present invention includes a general purpose computing system environment, such as computing system environment 100. Although specific components are illustrated in computing system 100, such components are exemplary. That is, apparatuses according to various embodiments may include various other components. It is further appreciated that not all of the components computing system 100 may be necessary for particular apparatuses.

In its most basic configuration, computing system environment 100 typically includes at least one processing unit 102 and memory 104. Depending on the exact configuration and type of computing system environment, memory 104 may be volatile (such as RAM), non-volatile (such as ROM, flash memory, etc.) or some combination of the two. This most basic configuration is illustrated in FIG. 1 by dashed line 106. Additionally, computing system environment 100 may also have additional features/functionality. For example, computing system environment 100 may also include additional storage (removable and/or non-removable) including, but not limited to, magnetic or optical disks or tape. Such additional storage is illustrated in FIG. 1 by removable storage 108 and non-removable storage 110. Computer storage media includes volatile and nonvolatile, and removable and non-removable media implemented in any method or technology for storage of information such as computer readable instructions, data structures, program modules or other data. Memory 104, removable storage 108 and non-removable storage 110 are all examples of computer storage media. Computer storage media includes, but is not limited to, RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by computing system environment 100. Any such computer storage media may be part of computing system environment 100.

Computing system environment 100 may also contain communications connection(s) 112 that allow it to communicate with other devices. Communications connection(s) 112 is an example of communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared (IR) and other wireless media. The term computer readable media as used herein includes both storage media and communication media. Computing system environment 100 may also have input device(s) 114 such as a keyboard, mouse, pen, voice input device, touch screen 124 or other touch input device, remote control receiver 126, microphone 128, etc. Output device(s) 116 such as a display 118, speaker(s) 120, printer, etc. may also be included. All these devices are well known in the art and need not be discussed at length here.

### Exemplary Systems and Methods for Administering and Analyzing the Results of, Opthalmic Examination, According to Various Embodiments

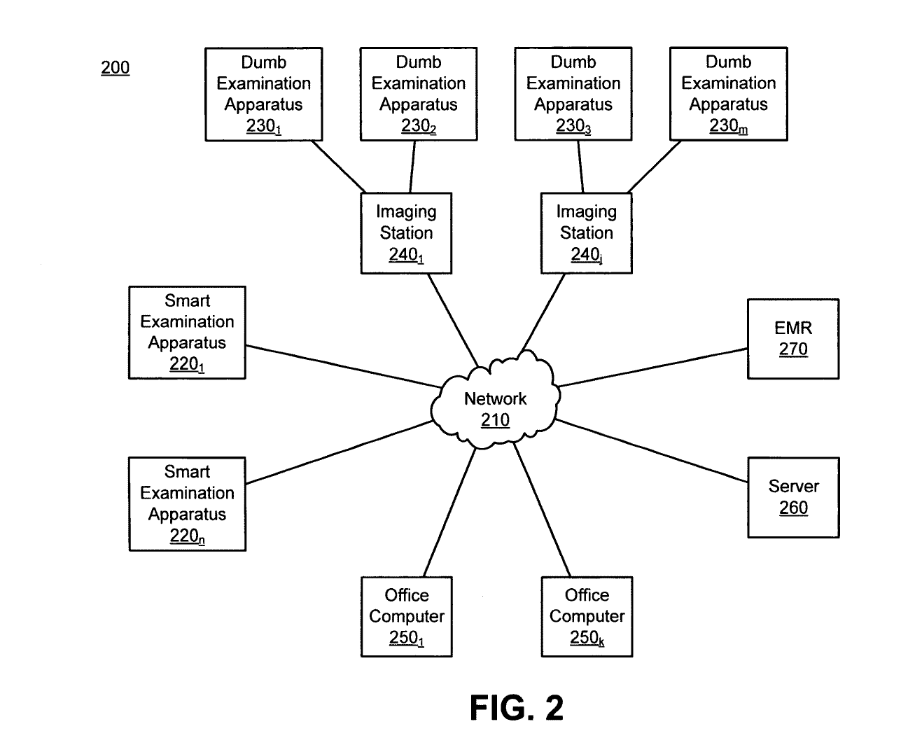

FIG. 2 illustrates a block diagram of a system 200 for administering, and analyzing the results of, ophthalmic examinations, in accordance with various embodiments of the present invention. System 200 is suitable for operation and communication over a computer network 210. Network 210 may be a local area network (LAN), a wide area network (WAN), the Internet, or any combination thereof.

System 200 includes one or more ophthalmic examination apparatuses 220, 230, such as an optical coherence tomographer, a retinal tomographer, a refractor, a keratometer, a perimeter, a fundus/retinal camera, a corneal topographer, an auto-refraction system, and/or a non-contact tonometer. However, it is appreciated that examination apparatuses 220 and 230 are not limited as such. Examination apparatuses 230 are “dumb examination apparatuses,” in that they communicate over network 210 by way of one or more imaging stations 240. Imaging stations may be general purpose computers, such as computing system 100 of FIG. 1, which, in the case of imaging stations 240, are also capable of interfacing with, and controlling, examination apparatuses 230. A single imaging station (e.g. station 240₁) may control multiple examination apparatuses (e.g. apparatuses 230₁, and 230₂).

Examination apparatuses 220 are “smart examination apparatuses,” in that they comprise computing systems, such as computing system 100, and they communicate directly over the network 210. In addition to the features described above with respect to FIG. 1, examination apparatuses 220 also include ophthalmic examination components 122, which include the hardware and/or software necessary for performing a particular ophthalmic exam.

System 200 may include one or more conventional, general-purpose office computers 250. System 200 may include an electronic medical record (EMR) database 270 for storing electronic patient records, including results of ophthalmic examinations conducted by apparatuses 220 and 230. System 200 further includes a server 260, which facilitates communications and data transfer between smart examination apparatuses 220, imaging stations 240, office computers 250, and EMR 270. It should be appreciated that instead of the server 260 and the EMR 270 being physically separate, as depicted in FIG. 2, EMR 270 may alternatively reside on server 260 itself.

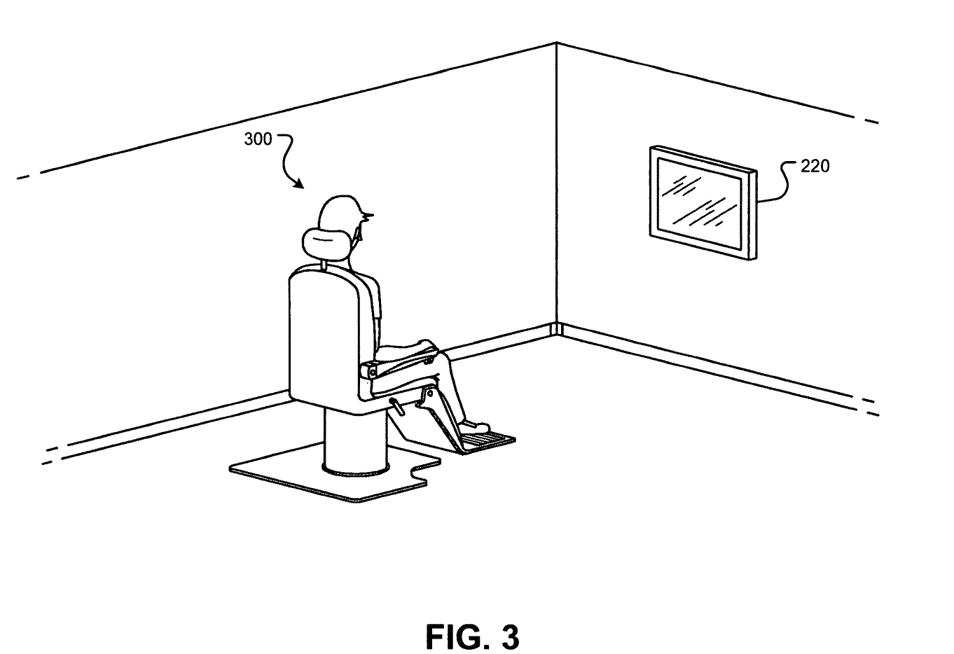

Particularly advantageous aspects of the present invention involve the smart examination apparatuses 220 discussed briefly above. In a preferred embodiment, the smart examination apparatus 220 is wall-mountable and includes a flat-panel display, such as an LCD display, a plasma display, or the like. As shown in FIG. 3, the apparatus 220 is preferably mounted on the wall that a patient 300 would normally face when being examined by an ophthalmic practitioner.

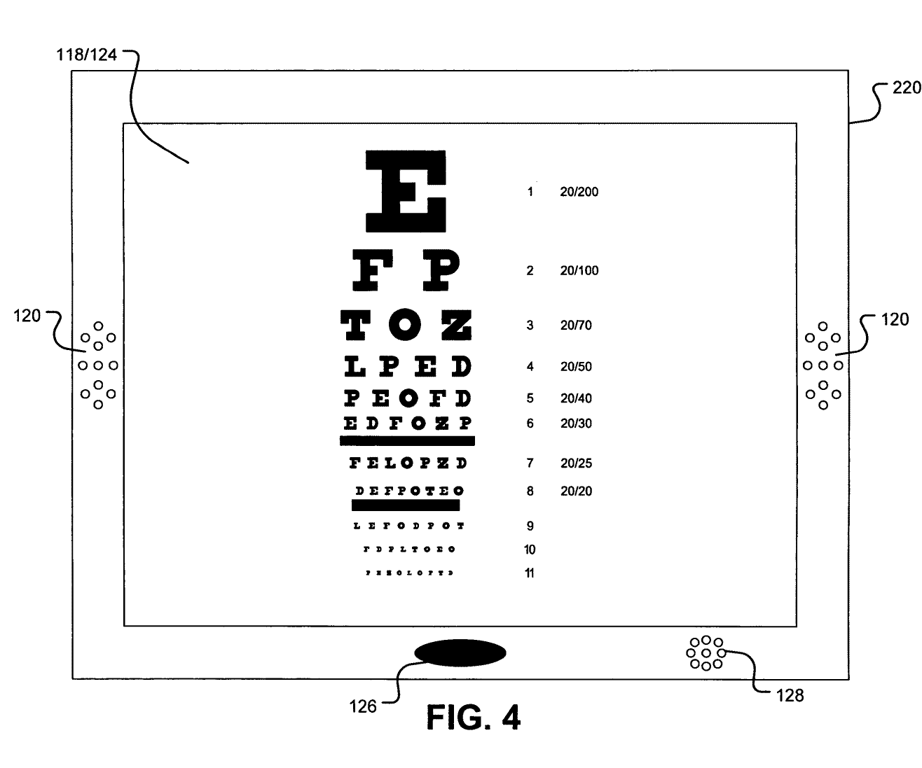

As discussed above, the smart examination apparatus 220 is operable to perform an ophthalmic examination. In a preferred embodiment, the ophthalmic exam is a visual acuity test, as illustrated in FIG. 4. In one embodiment, the smart examination apparatus 220 may perform the ophthalmic exam in response to an input received at an input device(s) 114. In the illustrated embodiment, the examination apparatus includes multiple input devices 114, including a touch screen 124 and a remote control receiver 126. The remote control receiver may be IR, RF, or any other conventional remote control technology. By way of example, a practitioner may press a predetermined button on a remote control to initiate the visual acuity test. The remote control input is received by the remote control receiver 126, upon which receipt the apparatus displays a visual acuity exam, such as the illustrated Snellen chart, on display 118. Alternatively, the practitioner may utilize the touch screen 124 to initiate the exam.

Either during or after the administration of the acuity test, the practitioner may wish to pull up the patient's results from a different ophthalmic exam that was previously performed taken by a separate examination apparatus (e.g. apparatus 230₁). In response to a second input received from the practitioner, the examination apparatus 220 switches from an examination mode to an analysis mode, so as to display the desired test results. Again, the second input may be received via the remote control receiver 126, the touch screen 124, or any other conventional input device (mouse, keyboard, etc.).

Upon switching to analysis mode, the smart ophthalmic apparatus 220 may access the EMR 270 through the server 260. For example, the server 260 may provide the smart ophthalmic apparatus 220 with the ability to query the EMR 270 for the desired exam results. Once the desired results have been found, the ophthalmic apparatus 220 may request the desired results from the server 260. In response to the request, the server 260 may in turn retrieve the requested results from the EMR 270 and provide them to the apparatus 220. In one embodiment, the smart opthalmic apparatus 220 and the server 260, as well as other components in system 200, such as the imaging stations and the office computers 250, exchange EMR data using an integrated clinical software environment, such as Medmont Studio from Medmont International Pty. Ltd.

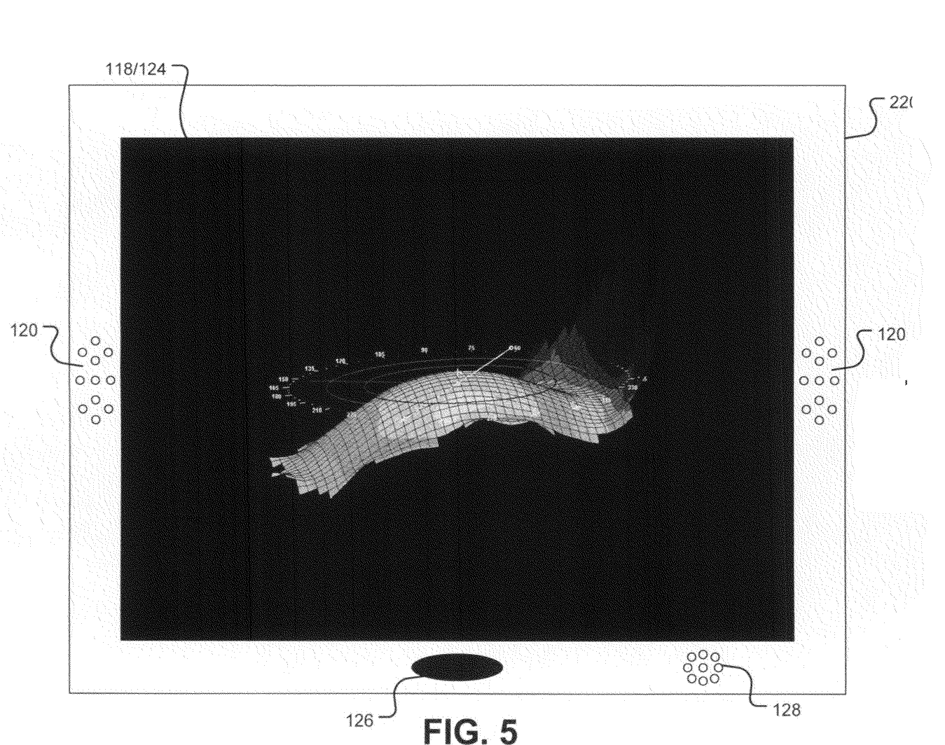

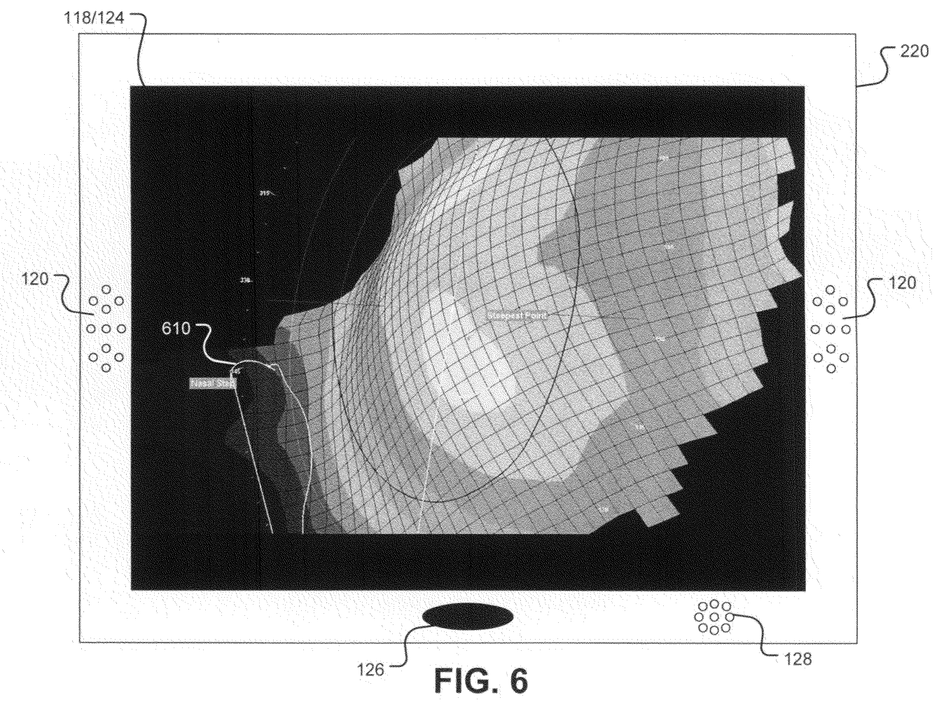

In the example shown in FIG. 5, the examination apparatus 220 is displaying the three-dimensional results obtained from a corneal topography exam. Significantly, the examination apparatus 220 is capable of displaying the results of a separate exam performed by a separate machine in a manipulatable and annotatable format. This may be achieved, for example, by displaying the subject test results in their native file format, rather than a static image (e.g. PDF, JPEG, etc.) of the results. Thus, rather than merely displaying a single view of three-dimensional results, various embodiments can display a fully-manipulatable version of the results, capable of being freely rotated. For example, FIG. 6 shows a rotated view of the three-dimensional test results of FIG. 5.

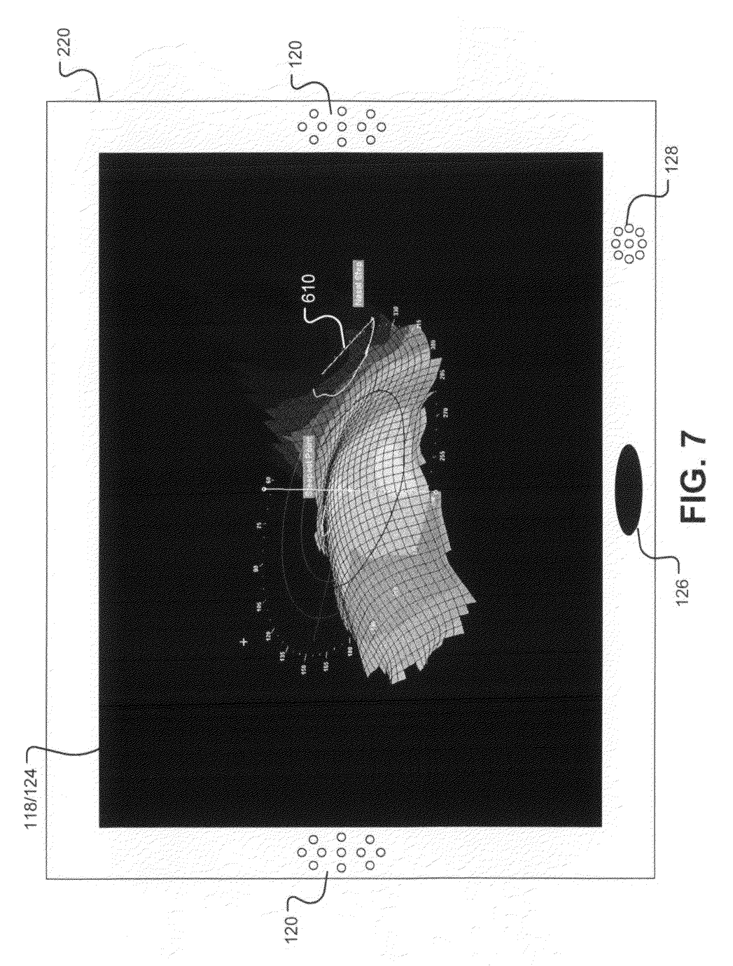

In addition to the ability to freely manipulate result data from ophthalmic examinations—and in particular, three-dimensional test data—various embodiments also enable the annotation of the test result data. As shown in FIGS. 6 and 7, the three-dimensional, topographical data displayed therein has been annotated to include free-form 610, which draws attention to an area of the image that a practitioner feels is significant. In one embodiment, the practitioner may use touch screen 124 to annotate the data, for example, by “drawing” the annotation on the touch screen 124. Either during the practitioner's annotation of the data, or upon the completion thereof, the result data in the EMR 270 may be updated to reflect the practitioner's annotations.

In addition to accessing, reviewing, manipulating, and annotating result data via a smart examination apparatus 220, system 200 also allows for such result data to also be manipulated and annotated in a similar manner via office computers 250. Thus, depending on a practitioner's preference, the practitioner can review and annotate test results either privately (i.e. on an office computer 250 in the practitioner's office) or in the presence of the patient on a smart examination apparatus 220. Moreover, because network 210 may include a WAN or even the Internet generally, a practitioner could review and annotate the exam results from virtually anywhere in the world that an internet connection in available.

Thus, various embodiments enrich the clinical experience for both the patient and the practitioner. First, the practitioner is not forced to keep the patient waiting while the practitioner reviews the patient's exam the exam results can be reviewed right in the exam room in the presence of the patient, and presented in a convenient, user-friendly, and full-featured manner. By allowing for full-featured, electronic annotation of exam results, clinic efficiency is enhanced, because physical access to a patient's file is not necessary each time a practitioner wishes to the patient's test results.

### Enhanced Features for Providing a Comprehensive Clinic Solution According to Various Embodiments

In addition to the foregoing, various embodiments also provide for other enhanced features toward providing a comprehensive clinic solution. For example, in one embodiment, the system 200 is capable of providing an intra-office intercom system across any compatible devices on network 210. As shown in FIGS. 4-9, the smart examination apparatus 220 may include a microphone 128 and one or more speakers 120. In one embodiment, in response to a particular input (e.g. from a remote control), the apparatus 220 may begin recording sound received at the microphone 128, during which time a user may speak a message into the microphone 128. When the apparatus 220 is finished recording the sound clip, the apparatus 220 may then transmit the sound clip to the server 260, which may in turn broadcast or “push” the sound clip to all compatible devices on the network 210 or, alternatively, to a selected subset of such devices. In an alternative embodiment, the recording apparatus 220 may bypass the server 260 and broadcast the sound clip to the other devices by itself.

In various embodiments, system 200 also provides a patient tracking system. This patient tracking system is particularly advantageous in offices employing multiple ophthalmic practitioners and/or utilizing multiple patient examination rooms. In these cases, it is beneficial for practitioners and other office employees to keep updated on which patient each practitioner is, or will be, examining, and in which examination room.

Accordingly, various embodiments are operable to cause an exam room status indicator to be displayed on the displays 118 of smart examination apparatuses 220, imaging stations 240, and office computers 250. In one embodiment, the status indicator appears on a display as a temporary pop-up window which is displayed for a predetermined amount of time (e.g. 30 seconds).

In other embodiments, the status indicator is displayed persistently. In yet other embodiments, the status indicator is displayed as a tab or panel within another application, such that it may be called up for viewing, or hid from view, as desired.

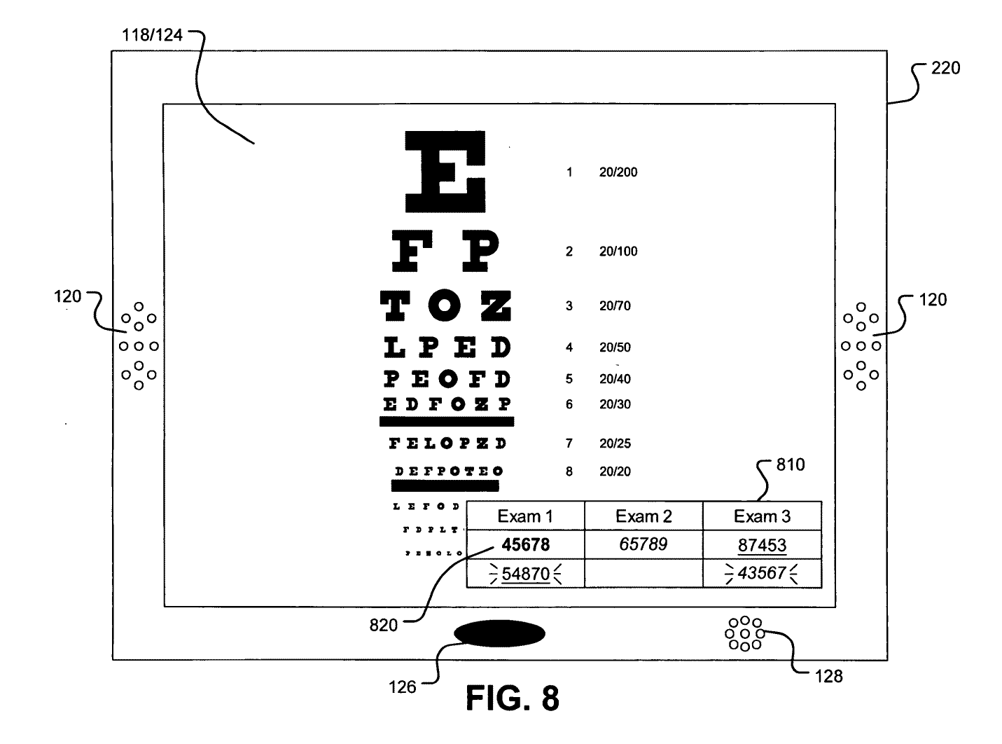

FIG. 8 illustrates the display of a exam room status indicator 810 on a smart examination apparatus 220, in accordance with various embodiments of the present invention. It should be appreciated that the size of status indicator 810 depicted in FIG. 8 has been exaggerated for the purpose of illustration. The status indicator 810 includes a plurality of patient indicators 820, which indicate which patient is in which exam room. This may be achieved in number of ways.

In the illustrated embodiment, a patient number is shown as being associated with a particular exam room. Specifically, the exam room status indicator 810 indicates that Patient No. 45678 is currently in Exam Room 1, that Patient No. 65789 is in Exam Room 2, and that Patient No. 87453 is in Exam Room 3. As an alternative to displaying patient numbers, the status indicator 810 may display the patients' initials or the patients' names, though this may be undesirable in terms of preserving patient confidentiality. Instead of the simple table format of FIG. 8, the status indicator 810 may alternatively be in the form of a graphical floor plan, which displays the layout of the exam rooms in a given office. The patient indicators 820 would then be displayed within the appropriate exam room.

In one embodiment, in addition to showing which patients are currently in each of the exam rooms, the status indicator 810 also indicates which patient is scheduled to be each exam room next. For example, in the illustrated embodiment of FIG. 8, the middle row of status indicator 810 indicates which patients are currently in the exam rooms, while the bottom row indicates which patients are scheduled to be in the rooms next.

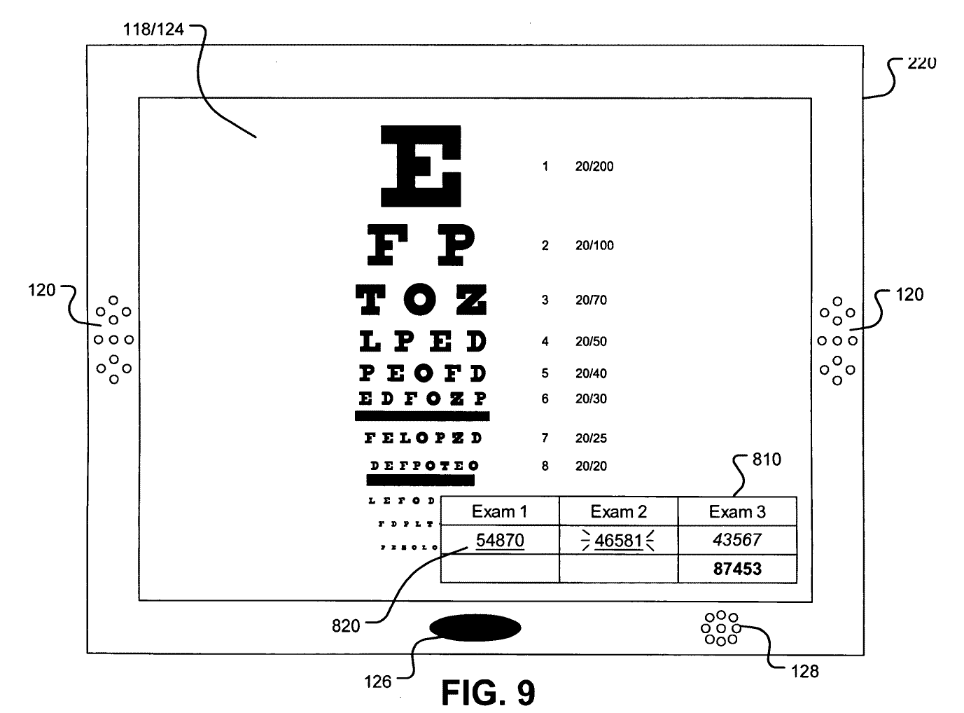

The exam room status indicator 810 may also indicate which practitioner is scheduled to see a particular patient. It should be appreciated that this may be achieved in a number of ways. In one embodiment, the patient indicators 820 may be color-coded by practitioner. For example, a red patient indicator 820 may correspond to Dr. X, a blue patient indicator 820 may correspond to Dr. Y, an orange patient indicator 820 may correspond to Dr. Z, etc. In the illustrated embodiment, an underlined patient indicator 820 corresponds to Dr. X, a bold-faced patient indicator 820 corresponds to Dr. Y, and an italicized patient indicator 820 corresponds to Dr. Z. In one embodiment, a patient indicator 820 corresponding to a patient that a practitioner is currently seeing is persistently displayed, while a patient indicator 820 corresponding to a patient that the practitioner is scheduled to see next may be flashing. For example, as shown in FIG. 9, Dr. X currently has patients in both Exam Room 1 and Exam Room 2. Dr. X knows that s/he should see Patient No. 54870 first because the number 54870 is solid, while the number 46581 is flashing (flashing indicated by dashed lines).

The exam room status indicator 810 is updated or “refreshed” as patients are cycled through the exam rooms. In one embodiment, a particular user, such as a receptionist or office manager, may use administrative software running on his or her office computer 250 to control and update the display of the status indicators 810 displayed on other terminals 220, 240, 250 throughout the office. For example, a receptionist may enter or edit the listing of which patients are currently in, and/or are scheduled to be in, the exam rooms. Once the receptionist has finished updating the listing, the updated listed is transmitted to server 260, which in turn pushes the update to the rest of the terminals. As shown in FIG. 8, Patient No. 45678 is currently in Exam Room 1, Patient No. 65789 is in Exam Room 2, and Patient No. 87453 is in Exam Room 3, while Patient Nos. 54870 and 43567 are the next scheduled patients to be in Exam Rooms 1 and 3 respectively. FIG. 9 shows the status indicator 810 after a subsequent update. Notably, Patient Nos. 54870 and 43567 have been updated from a “next-in-room” status in Exam Rooms 1 and 3, respectively, to a “current” status. Additionally, Patient No. 45681 has been added to status indicator 810 as currently in Exam Room 2, and Patient No. 87453 has been added as “next-in-room” for Exam Room 3.

Thus, various embodiments provide an “at-a-glance” indicator which allows for clinic employees to stay updated on which patients are in which exam rooms, and which practitioners are scheduled to see those patients. Moreover, because status updates can be provided via a server-push and may appear as a temporary pop-up window, clinic employees do not have to constantly log in and/or refresh their displays to receive status update.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A computer-implemented method of administering, and analyzing the results of, ophthalmic examinations, the method comprising:
generating a display on a display device to enable a first ophthalmic exam to be administered;
receiving a request for results of a second ophthalmic exam;
requesting the results of the second ophthalmic exam from a data source;
receiving the results of the second ophthalmic exam from the data source;
displaying the results of the second ophthalmic exam on the display device; and
enabling annotation or modification of the results displayed on the display device.

2. The method as recited in claim 1, wherein displaying the results of the second ophthalmic exam on the display device comprises displaying a graphical representation of the results of the second ophthalmic exam on the display device.

3. The method as recited in claim 1, further comprising:
updating the data source to reflect the annotation or the modification of the results of the second ophthalmic exam.

4. The method as recited in claim 1, wherein the data source comprises an electronic medical record (EMR) database.

5. The method as recited in claim 1, wherein displaying the first ophthalmic exam on the display device comprises displaying a visual acuity chart on the display device.

6. The method as recited in claim 1, further comprising:
receiving an input from an ophthalmic practitioner, and
wherein displaying the first ophthalmic exam on the display device comprises displaying the first ophthalmic exam in response to receiving said input.

7. The method as recited in claim 1, wherein receiving the request for results of the second ophthalmic exam comprises receiving an input from an ophthalmic practitioner.

8. The method as recited in claim 1, wherein enabling the annotation or the modification of the results displayed on the display device comprises providing a touch-sensitive region on the display device.

9. The method as recited in claim 1, wherein enabling the annotation or the modification of the results displayed on the display device comprises enabling the annotation or the modification of the results displayed on the display device by an ophthalmic practitioner.

10. The method as recited in claim 1, wherein the results of the second ophthalmic exam comprise three-dimensional image data, and wherein further displaying the results of the second ophthalmic exam on the display device comprises displaying a rotatable visual representation of the three-dimensional image data on the display device.

11. The method as recited in claim 1, further comprising:
receiving an input via an input device;
recording a sound clip in response to the input; and
providing the sound clip to another computer for playback.

12. The method as recited in claim 1, further comprising:
displaying on the display device an exam room status indicator, wherein the exam room status indicator indicates which patient is currently in a particular exam room.

13. The method as recited in claim 12, wherein the exam room status indicator further indicates which patient is scheduled to be in the particular exam room next.

14. The method as recited in claim 12, wherein the exam room status indicator further indicates which ophthalmic practitioner is assigned to the patient currently in the particular exam room.

15. The method as recited in claim 12, further comprising:
updating the exam room status indicator to reflect occupancy of the particular exam room by a different patient.

16. The method as recited in claim 15, wherein updating the exam room status indicator comprises:
receiving an exam room status update notification; and
updating the exam room status indicator based on the exam room status update notification.

17. A system for administering, and analyzing the results of, ophthalmic examinations, the system comprising:
a server communicatively coupled with a database having results of ophthalmic examinations stored therein; and
an ophthalmic examination apparatus communicatively coupled with the server, the ophthalmic examination apparatus comprising a display and an input device,
wherein the ophthalmic examination apparatus is operable to perform a first ophthalmic examination,
wherein further the ophthalmic examination apparatus is operable to retrieve results of a second ophthalmic examination from the data server and display the results of the second ophthalmic examination on the display, and
wherein further the ophthalmic examination apparatus is operable to annotate or modify the results of the second ophthalmic examination in response to an input received at the input device.

18. The system as recited in claim 17, wherein the ophthalmic examination apparatus is a first ophthalmic examination apparatus and the system further comprises:
a second ophthalmic examination apparatus communicatively coupled with the server,
wherein the second ophthalmic examination apparatus is operable to perform the second ophthalmic examination.

19. The system as recited in claim 18, further comprising:
an imaging station communicatively coupled with the second ophthalmic examination apparatus, wherein the imaging station is operable to control the second ophthalmic examination apparatus.

20. The system as recited in claim 17, wherein the database comprises a electronic medical records (EMR) database.

21. The system as recited in claim 20, wherein the EMR database resides on said server.

22. The system as recited in claim 20, wherein the EMR database resides on a machine separate from said server.

23. The system as recited in claim 17, wherein the results of the second ophthalmic examination comprise three-dimensional image data, and wherein further the ophthalmic examination apparatus is operable to display a rotatable visual representation of the three-dimensional image data.

24. The system as recited in claim 23, wherein the ophthalmic examination apparatus is operable to annotate or modify the three-dimensional image data in response to the input received at the input device.

25. The system as recited in claim 17, wherein the first ophthalmic examination comprises a visual acuity examination.

26. The system as recited in claim 17, wherein the second ophthalmic examination is selected from the group consisting of an optical coherence tomography examination, a retinal tomography examination, a refraction examination, a keratometry examination, a peripheral vision examination, a fundus examination, a retinal examination, a corneal topography examination, an auto-refraction examination, a gonioscopy examination, a retinoscopy examination, and a non-contact tonometry examination.

27. The system as recited in claim 17, wherein the display comprises a flat-panel display.

28. The system as recited in claim 17, wherein the input device comprises a touch-screen.

29. The system as recited in claim 17, wherein the input device comprises a remote control.

30. The system as recited in claim 17,
wherein the ophthalmic examination apparatus further comprises a microphone,
wherein further the ophthalmic examination apparatus is operable to record a sound clip with the microphone and transmit the sound clip to the server, and
wherein further the server is operable broadcast the sound clip over a data network to one or more other computerized devices for subsequent playback.

31. The system as recited in claim 17, wherein the ophthalmic examination apparatus is operable to display on the display device an exam room status indicator indicating which patient is currently in a particular exam room.

32. The system as recited in claim 31, wherein the exam room status indicator further indicates which patient is scheduled to be in the particular exam room next.

33. The system as recited in claim 31, wherein the exam room status indicator further indicates which ophthalmic practitioner is assigned to the patient currently in the particular exam room.

34. The system as recited in claim 31, wherein the ophthalmic examination apparatus is operable to update the exam room status indicator to reflect occupancy of the particular exam room by a different patient.

35. The system as recited in claim 34,
wherein the server is operable to provide an exam room status update notification to the ophthalmic examination apparatus, and
wherein the ophthalmic examination apparatus is operable to update the exam room status indicator based on the exam room status update notification.
